(19) United States
(12) Patent Application Publication
(10) Pub. No.: US 2004/0019262 A1
(43) Pub. Date: Jan. 29, 2004

(54) 3 dimensional imaging of hard structure without the use of ionizing radiation

(76) Inventor: Michael D. Perelgut, Thornhill (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/200,442

(22) Filed: Jul. 23, 2002

(51) Int. Cl. A61B 5/05

(52) U.S. Cl. 600/407

(57) ABSTRACT

A diagnostic process for generating, recognizing, and remotely examining layers of tooth using processed reflection data from physical waves to produce high-resolution quantitatively measurable 3D images. The present invention examines interior portions of tooth structure. The layers can be considered to be common impedance objects, which are present in a uniform background. Acquire data sets for the area of interest and then acquire a 3 dimensional reflection data volume. This data is then subjected to diagnostic 3 dimensional processing to produce a vertical and horizontal high-resolution matrix. In a similar manner this method of imaging tooth structure can be used to measure other hard structures in the body (i.e. bone) or outside the body (i.e. cement, concrete, rock etc).

### FIELD OF THE INVENTION

This invention relates generally to the imaging of 3 dimensional hard structures. More specifically this invention relates to the 3 dimensional imaging of dental structures. Secondary.

### BACKGROUND OF THE INVENTION

In the field of dentistry there is a need for viewing the internal structures of teeth in order to diagnose most dental pathology definitively. At present the only way to view any internal structures of teeth is with radiology. The present field of dental radiology has 2 major drawbacks.

One is that the process is based on ionizing radiation that penetrates human tissue and the amount of energy that is not absorbed by such tissue is transferred to a receiver (film, sensor, et al). Ionizing radiation has been implicated in many serious medical pathologies. Modern medicine recognizes that it should be avoided or minimized if possible.

The second is that the image is a 2 dimensional representation of a 3 dimensional image. This severely limits their diagnostic effectiveness. There are presently methods of doing 2 dimensional slices of the jaw. This method gives poor quality pseudo 3 dimensional views.

By using a physical wave source and evenly spaced sensors placed on tooth structure it is possible to generate a 3 dimensional image of the tooth. The theory is based on the present methods used by the global seismology to map the internal structures of the earth. This method deals with the determination of the earth's internal structures using earthquake induced seismic waves. With sensors placed on the surface of the earth at distances of 1000s of kilometers measurements of the incoming wave patterns verses time will give data that can interpret the level at which the next change in rock density occurs.

The oil and gas industries have taken these methods to another area. The object of the oil and gas industry is to determine where pockets of nonsolid structures are located within the earth. The 3dimensional image used in the Oil and gas industry is done by producing suitable size ‘explosions’ on the surface of the earth at different positions while keeping the sensors constant. By ‘stacking’ the data obtained, a 3 dimensional image can be formed.

Discussion of common method of analyzing data from geophysical and oil/gas data as discussed.

Discussion of transferring present methods on the scale of 1000 kilometers to a scale of 10 mm.

Discussion of sensor placements and limitations. Use of a uniform injectable material for 1st layer sensor placement.

In dentistry an accurate 3 dimensional image of a tooth can be invaluable. It can be utilized in all the specialty areas of the dental field:

Endodontics: The 3 dimensional image can give the practitioner the precise location of internal canal system of the tooth. This can include the exact location of horizontal fractures, vertical fractures, the number of canals, the presence of accessory canals, the presence of nutrient orifices, the height of canals in comparison to prostheses, the final fill and quality of obturation, et al.

Periodontics: The ligament attachment of periodontal tissue is imbedded into the cementum of tooth. The presence of these insertions can be precisely determined and thus give an accurate description of the periodontal condition of the dentition.

Oral surgery: With the extension of this invention into the imaging of bone the practitioner will be able to determine precise location of landmarks, location of pathology, get a quantitative measurement of bone quality, et al

Prosthodontics: The three dimensional image of the tooth can be used to determine endodontic limitations, get an exact 3dimensional image of the tooth prior to preparation and a digitized ‘impression’ of the tooth for restoration.

Orthodontics: Periodontal condition of the dentition, external and internal resorptions, presence of landmarks and pathology

This 3 dimensional imaging of the tooth can be expanded to include ‘automatic’ preparation/restoration of tooth structure. By using the “rule” of tooth restoration (regardless of choice of restoration) if the external, internal, occlusal, and functional information for a persons dentition is know, then an ideal preparation can be made to minimize the amount of tooth structure removed and subsequent prostheses to replace the removed structure can be made external to the patient concurrently thus eliminating some of the limiting factors involved in restoring form and function to the dentition.

### ALTERNATE DESCRIPTION

By applying a physical wave (seismic wave) to a solid object with distinct internal boundaries, we can measure the time it takes for the wave to reflect off those boundaries and the angle at which they arrive at the surface. The physical wave can be divided into different types based on orthogonality. The first wave type of interest is the P wave; the second is the S wave. Let us first describe the P wave. As it passes the first boundary, part of the wave is reflected and part is transmitted. This first part, which is reflected, can be measured at a distant spot. As the wave passes to a second boundary with in the solid, again part of the wave is reflected and part is transmitted. This continues throughout the solid. Each reflection has a certain signature, which can be used to determine which wave is arriving at the receiver. This theory is similar to the global model, which has been used throughout modern global examinations of the earth's interior. The major differences in the earth model and the tooth model is 1) the density of the layers of tooth are well known and 2) the size of the earth (˜10000 Km) and the size of the tooth (˜10 mm) 3) the global shape of the earth and the different surface shape of the tooth. Please see attached publications on the mathematical methods described in global seismology to describe the measurements of the layers of the internal parts of the earth.

The first is an advantage to the measurement of the tooth. The knowledge of the density of the tooth layers will in turn tell us the relative speed of the wave through that object. This in turn eliminates some of the variables in the equation.

The second is a disadvantage in that when the size of the object is lessened (in this case considerably) the energy of the wave needs to be increased. The energy levels needed (i.e. wavelengths) are well within an achievable range.

The third is controllable in different ways. The first is by adding a coupling material as the first layer. The second is by getting the external shape of the tooth imaged and mathematically adjusting the results.

This entire method can be transferred to the bone as opposed to the tooth itself. This will give us the image of the bone itself. As well this technique can be transferred to any solid layered object.

### TECHNICAL BACKGROUND

The determination of the external and/or internal structure of a solid object is desired in a wide field of technical applications because it is of special interest to get information about an object without destroying it. Many apparatuses and methods are known for this purpose. Specifically in the medical field it is an advantage to get the best information of the interior of the human body without having to be invasive.

### PRESENT METHODS (STATE OF THE ART)

The most common and widely used method for determining hard structure in the living body is x-ray technology. Other such methods could include the use of lasers reflection and refraction of light to determine the depth of the change in dental structure. The method will prove useful should the energy level and detection of the light be detectable. Since lasers are becoming mainstream in the use of medicine and dentistry, the use of lasers for measurement is a logical next step.

It is known from geophysical data acquisition, processing and imaging techniques to get information regarding the internal structures in the earth. The interpretation of P and S seismic waves from a single source or a number of sources is described in U.S. Pat. Nos. 4,363,113 4,072,922 4,259,733 5,153,858 5,671,136 5,018,112 5,586,082 et al. These patents describe methods that are employed after data acquisition is completed and all methods are numerical and computational in nature.

It is also known from global seismology that the internal structure of the earth can be measured following large seismic events and spaced receivers. By using the same well known computations we can determine the layers at which the boundaries in change of tooth structure can occur. This method uses the S and P wave calculations commonly in use in the science of seismology.

### BASIC DESCRIPTION OF PHYSICS

1. A Method of obtaining, from data received from transmitted physical waves into subsurface dental layers and receiving reflected seismic signals from formations with a line of detectors uniformly over an area greater than the 1^{st} Fresnel zone for waves.

2. Repeating the above step for a plurality of parallel lines of profile

3. Sorting results based on transversity to lines of profile

4. Migrating sections to get 3 dimensional data

5. Repeating the steps for delayed wave fronts

Traces synthesizing the response of intradental substructure density changes (DEJ, CDJ, etc) to cylindrical or plane waves are obtained for a succession of shot point locations along a line of profile. The traces obtained are then shifted to produce the effect of a steered or beamed wave front and the steered traces and original trace for each shot point are summed to form synthesized trace for a beamed wave front. The synthesized traces are then collected into sets are assembled to form a plurality of synthesized sections, beamed vertically downward (or other directions). A number of these sections are then individually imaged or migrated, and the migrated sections are summed to form a migrated 2-dimensional stack of data from cylindrical or plane wave exploration. Reflectors are located correctly in the in-line direction. The traces for shot points of the lines which are perpendicular to these lines are then assembled and processed to obtain a 3-dimensional migrated image.

Principles: Using waves generated by individual surfaces sources positioned on the tooth 3 dimensional reflection surveys can be generated. Separate digital recordings are then made by multiple receivers following each vibration sweep. Based on Huygens' principle (successive wave fronts acting as a source for new wavefronts) a sophisticated computerized process can be developed to model the arrival times seen on recorded traces from each intradental tooth reflecting alyer. This can be modeled after the exploding reflector model in seismology. This data can be processed using the 3 dimensional migration theory.

### DESCRIPTION OF INVENTION

To overcome the inconveniences of existing technologies, the invention proposes an apparatus for determination of internal and/or external tooth structure of a solid object, especially for medical, dental or civil engineering objects, comprising a wave generating source, a wave receiver and a signal evaluation unit, characterized in that there are at least two receivers spaced apart, in that the source can be placed at a first position and possibly to numerous other positions at known distances apart.

A further object of the invention is a method for determination of the external and/or internal structure of solid objects, especially for medical and dental objects, where in a first step at least one wave generating source and at least two wave receivers are placed at or nearby the object, that in a second step a first seismic wave is emitted by the source and received by the receivers whereby the wave has traveled through the object by seismic wave propagation, that in a third step a second wave is emitted by the source and received by the receivers whereby the wave has traveled through the object by seismic wave propagation This process is repeated an adequate number of times delivering a set of received signals.

It is advantageous to use the first arrival travel time generation for determination of external structure.

For determination of the internal structure it is advantageous to use the full waveform imaging.

The use of seismic waves of frequencies between 10 MHz and 250 MHz (preferably 40 MHZ to 50 MHz) are used to determine structures in the order of 10 mm in diameter compared to those in the order geophysical (1000 km to 10000 km).

### EXAMPLES

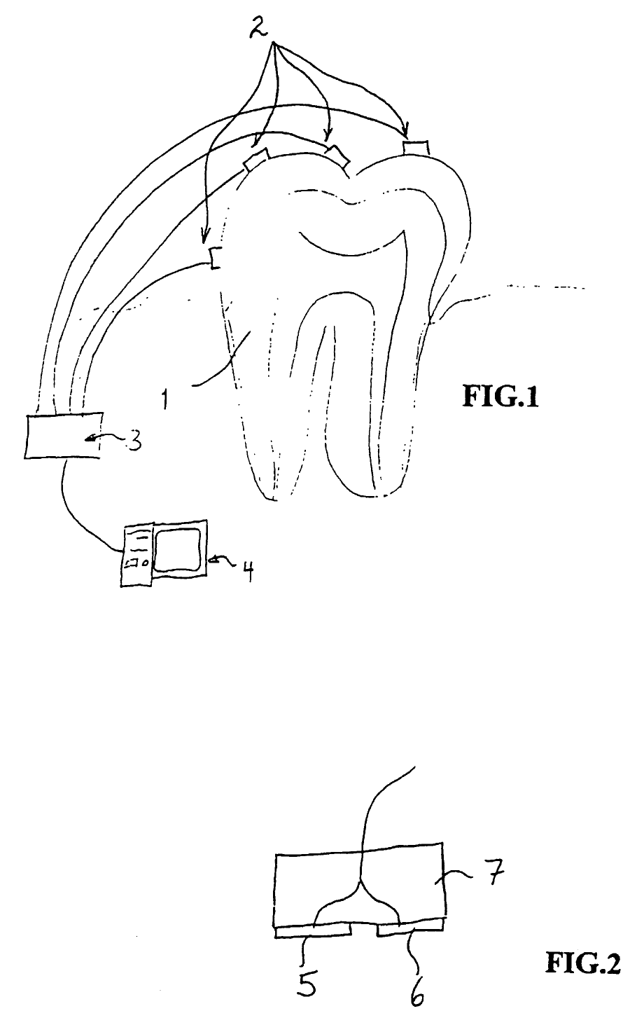

FIG. 1. An apparatus for determination of the external and internal structure of a tooth 1 with dimensions less than 2 cm in every direction as an example for tooth structure. At or nearby the tooth 1 are placed multiple sensors 2 connected to a unit to collect the data 3 computer 4 to evaluate the signal. The signal evaluates the S and/or P seismic wave formations from direct and internal reflections/refractions. By placement of numerous sensors and using conventional stacking computations, an image of the internal layers and anomalies of the tooth can be visualized.

FIG. 2 the sensors 2 are comprised of a wave-generating source 5 and a wave receiver 6, both located in the same body 7 or located at different positions. For a resolution of ˜50 microns and a structure size of ˜2 cm a frequency of ˜40 MHz to ˜50 MHz source is used. However the frequencies can vary from ˜10 MHz to ˜250 MHz.

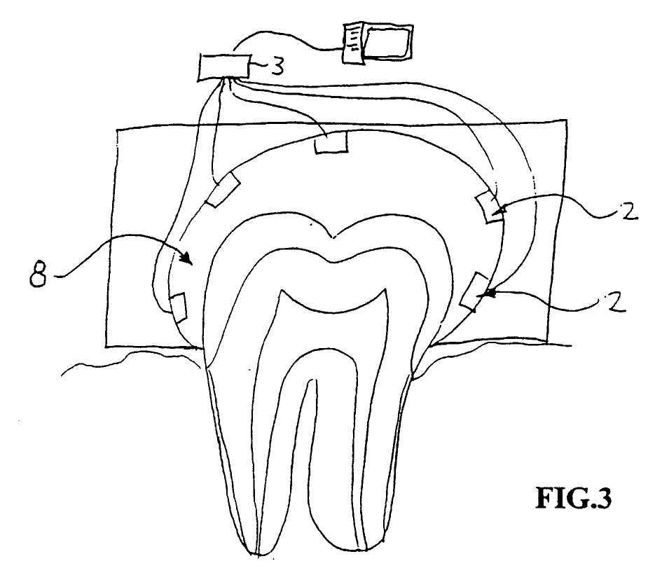

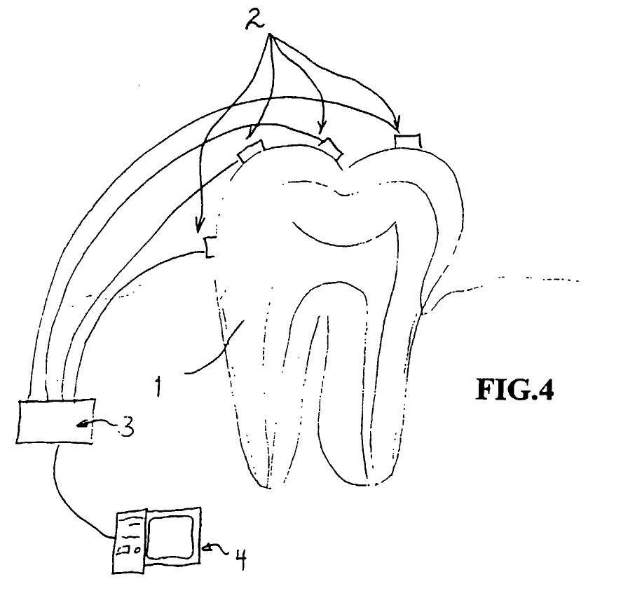

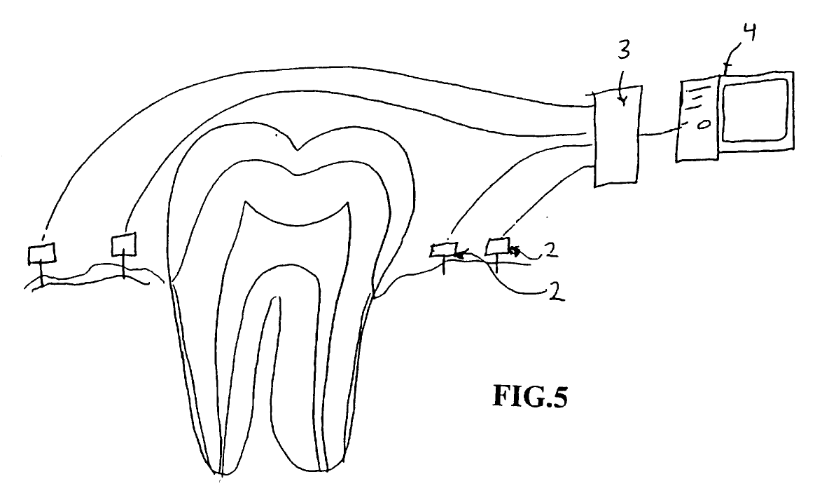

FIG. 3 the sensors are embedded in a uniform hard substance 8 which can be injected (i.e. acrylic, resin, stone or other material). The receiver 6 comprises the means for the measurement of the displacement in a vertical and/or horizontal direction. The material 8 surrounds the clinical crown of the tooth. The sensors 7 are spaced evenly and this uniform spacing is taken into account in the manipulation of the acquired data at the computer 4.

FIG. 4 Alternatively the sources 2 and receivers 2 are placed on the tooth structure.

FIG. 5 Similarly the Source/receivers 7 can be placed directly on the bone 8 using an acupuncture (or similar) technique. With 2 or more source/receiver combinations an image of the bone can be realized.

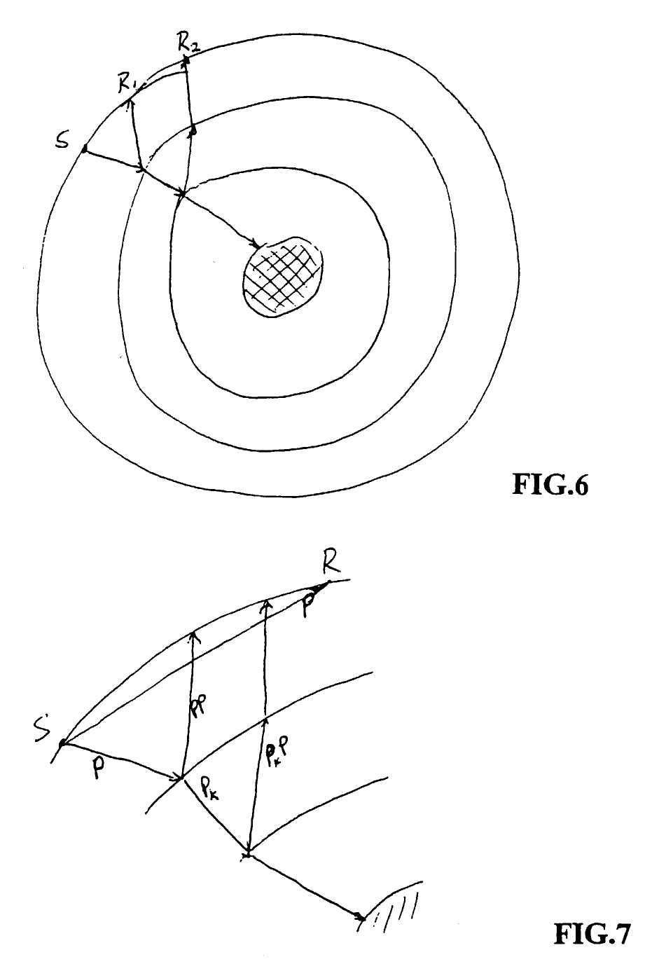

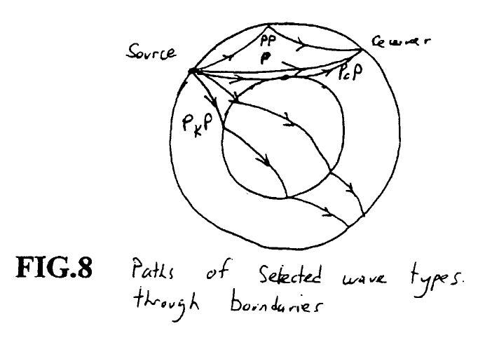

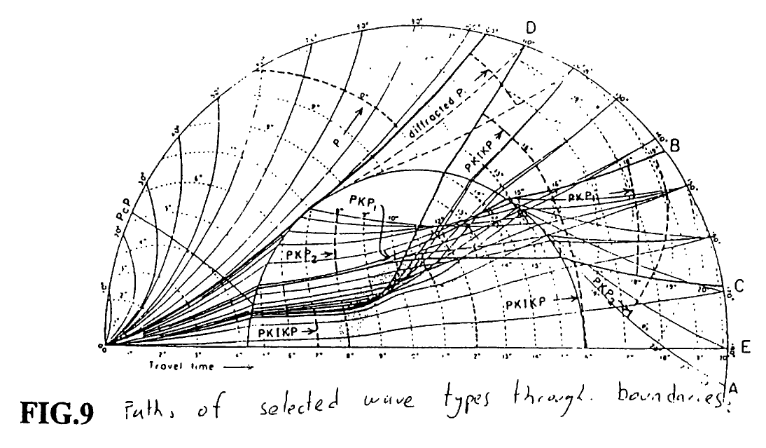

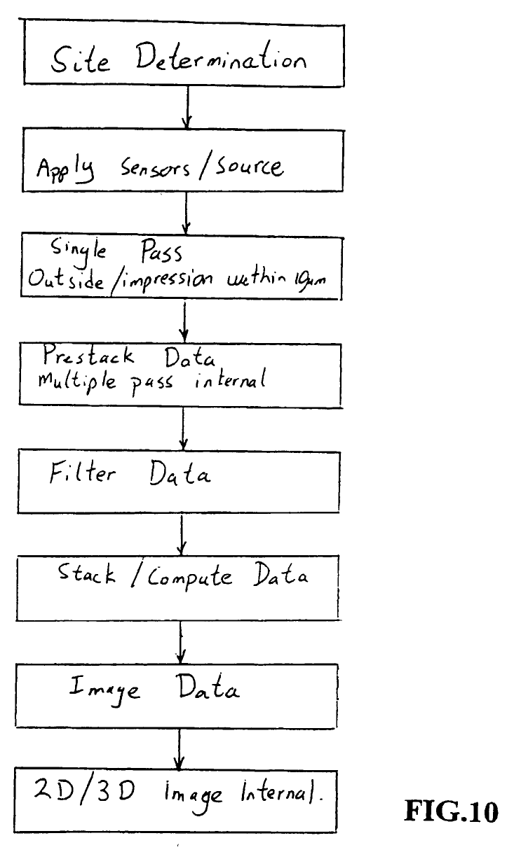

FIG. 6 Similarly the source/receivers 7 can be placed on any hard structure of any size (bridges, buildings, etc) and the source amplitude (and frequency) can be changed appropriately.

### SUMMARY OF THE INVENTION

Briefly the present invention provides a new and improved method for imaging the internal and external structures of the tooth. By eliminating the need for ionizing radiation, a safer, more effective method of imaging dental, medical and related hard structure can be obtained. As well this technology can be expanded to encompass other areas not related to dentistry and/or medicine.

The embodiments of the invention in which an exclusive property or privilege is claimed are defined as follows:

1. A method of performing seismic survey on a layered solid object
a) Placing sources and receivers on the external surface of the object. Each of these source receivers having a plurality of regularly spaced source/receiver stations, each receiver station adapted to detect seismic signals,
b) Inducing seismic signals into the solid object; and
c) Recording seismic signals detected by the receiver stations.
d) obtaining separate measures of compressional and shear wavefields incident on reflecting interfaces in the object's subsurface;
e) obtaining measures of compressional and shear wavefields scattered from the reflecting interfaces with in the object;
f) producing time-dependent reflectivity functions representative of the reflecting interfaces from the compressional and shear wavefields incident thereon and the compressional and shear wavefields scattered therefrom; and
g) migrating the time-dependent reflectivity functions to obtain depth images of the reflecting interfaces in the object's subsurface.

2. The method of claim 1 wherein the source and receivers are placed separately along the surface of the object.

3. The method in claim 2 where the receivers pick up the initial/external wave associated with the surface of the object.

4. The method in claim 3 where that external information is converted to an image.

5. The method in claim 3 where that information is used as a base to image the internal aspects of a layered object.

6. The method in claim 1 where the internal aspects of an object are imaged using 2 or more sources and/or receivers on the surface of the object.

7. The method in claim 6 where the internal aspects of a layered object are imaged using 2 or more sources and/or receivers.

8. Method in claim 1 where the depth of the surface area of a liquid portion of an object can be determined and imaged.

9. Method in claim 1 where the multiple layers of a layered solid object can be determined to a resolution of 100 microns or less.

10. Method in claim 1 where the multiple layers of a layered solid object can be determined to a resolution of 50 microns or less.

11. Method in claim 1 where the multiple layers of a layered solid object can be determined to a resolution of 10 microns or less.

12. Method in claim 1 where the multiple layers of a layered solid object can be determined to a resolution of 1 kilometre or less.

13. Method in claim 1 where the multiple layers of a layered solid object can be determined to a resolution of 0.1 kilometer or less.

14. Method in claim 1 where the multiple layers of a layered solid object can be determined to a resolution of 1 metre or less.

15. Method in claim 1 where the object consists of dental structure.

16. Method in claim 15 where the object is specifically a tooth.

17. Method in claim 16 where the external surface of the tooth is imaged.

18. Method in claim 16 where the internal layers of a tooth are imaged.

19. Method in claim 16 where 2 or more sources and receivers located at the same location or at different locations on the tooth surface image the internal structure of the tooth.

20. Method in claim 16 where 2 or more sources and receivers located at the same location or at different locations within a substrate on the tooth surface images the internal structure of the tooth and the surface of the tooth.

21. Method in claim 15 where 2 or more sources/receivers are placed on the bone to image the external surface of the bone.

22. Method in claim 15 where 2 or more source/receivers are placed on a solid object to image the layers of that object.

23. Method in claim 1 where the measurements are that of both P waves and/or S waves.

24. Method in claim 1 where a signal analysis devise processes the data to form a stacked or non stacked data set which in turn is then processed to form a 3 d computer image.

25. Method in claim 16 where the information can then be connected to a computer aided design and manipulation unit to prepare tooth structure for a restoration by:
a) Dynamically imaging the internal structure of the tooth in three dimensions.
b) Using the 3 dimensional image of the internal structure of the tooth and conventional or non-conventional preparation design to perform dental surgery on the tooth.

26. The method of claim 1 wherein the step of obtaining separate measures of the compressional and shear wavefields incident on the reflecting interface comprises obtaining separate measures of the compressional and shear wavefields for seismic energy imparted into the object's subsurface by seismic sources and the step of obtaining measures of the compressional and shear wavefields scattered from the reflecting interfaces comprises partitioning a set of multicomponent seismic data recording the object's response to seismic energy imparted into the earth's subsurface by the seismic sources to form reflected compressional and shear wavefields.

27. The method of claim 1 wherein the step of producing time-dependent reflectivity functions representative of reflecting interfaces includes separately cross-correlating the compressional and shear wavefields incident on reflecting interfaces with the compressional and shear wavefields scattered from the reflecting interfaces.

28. The method of claim 1 wherein the step of migrating the time-dependent reflectivity functions representative of the reflecting interfaces includes iteratively assuming velocities of propagation for the incident and scattered compressional and shear wavefields.

30. A method of imaging multicomponent seismic data to obtain depth images of the object's subsurface structures, comprising the steps of:
a) beam forming the multicomponent seismic data into sets of plane wave seismograms;
b) partitioning the plane wave seismograms into sets of compressional and shear wavefield seismograms;
c) forming time-dependent reflectivity functions from the sets of compressional and shear wavefield seismograms; and
d) migrating the time-dependent reflectivity functions to obtain depth images of the object's subsurface structures.

31. The method of claim 30 wherein the step of beam forming the multicomponent seismic data includes forming sets of plane wave seismograms for a plurality of beamed angles.

32. The method of claim 31 wherein the step of partitioning the sets of plane wave seismograms includes forming sets of compressional and shear wavefield seismograms for the plurality of beamed angles.

33. The method of claim 32 wherein the step of forming time-dependent reflectivity functions includes forming a plurality of reflectivity functions for the plurality of beamed angles.

34. The method of claim 33 wherein the step of migrating the time-dependent reflectivity functions includes migrating the time-dependent reflectivity functions for each of the plurality of beamed angles and stacking the migrated time-dependent reflectivity functions for the plurality of beamed angles to form depth images of the object's subsurface structures.

35. A method for imaging the object's subsurface structures, comprising the steps of:
a) collecting a set of multicomponent seismic data with seismic sources having at least one linearly independent line of action and receivers having at least two linearly independent lines of action;
b) sorting the set of multicomponent seismic data into incident angle ordered gathers;
c) partitioning the incident angle ordered gathers of the set of multicomponent seismic data into compressional and shear wavefields; and
d) migrating the compressional and shear wavefields to obtain a depth image of the object's subsurface structures.

36. The method of claim 35 wherein the step of sorting the set of multicomponent data includes the step of beam forming the set of multicomponent seismic data for a plurality of beamed angles.

37. The method of claim 36 further including the steps of:
a) transforming the set of multicomponent seismic data into the frequency domain;
b) partitioning the frequency domain set of multicomponent seismic data into a plurality of wavefield potentials; and
c) transforming the plurality of compressional and shear wavefields to the time domain.

38. The method of claim 37 wherein the step of partitioning includes forming a plurality of compressional and shear wavefields incident upon reflecting interfaces in the earth's subsurface and resulting compressional and shear wavefields scattered from the reflecting interfaces.

39. The method of claim 38 further including the step of cross-correlating the incident and scattered compressional and shear wavefields to form time-dependent reflectivity functions representative of reflecting interfaces in the object's subsurface.

40. The method of claim 39 wherein the step of migrating the compressional and shear wavefields includes migrating the time-dependent reflectivity functions to obtain depth images of the object's subsurface structures.

41. The method of claim 40 further including the step of stacking the plurality of migrated compressional and shear wavefields to form depth images of the object's subsurface structures.

42. A method for imaging the object's subsurface structures, comprising the
a) collecting a set of multicomponent seismic data;
b) partitioning the set of multicomponent seismic data so as to separate and decouple compressional and shear wavefield potentials in the set of multicomponent seismic data;
c) iteratively migrating the separated and decoupled compressional and shear wavefields for a plurality of assumed compressional and shear interval velocities; and
d) selecting from the plurality of assumed compressional and shear wave and shear interval velocities, the compressional interval velocities which produce coherent migrated wavefields.

43. The method of claim 41 wherein the step of partitioning includes obtaining a measure of the compressional and shear wavefields incident upon reflecting interfaces and resulting compressional and shear wavefields scattered therefrom.

44. The method of claim 42 further including the step of cross-correlating the compressional and shear wavefields incident and scattered from reflecting interfaces to obtain reflectivity functions representative of the reflecting interfaces.

45. The method of claim 43 wherein the step of iteratively migrating the compressional and shear wavefields includes iteratively migrating the shear and compressional wavefields of the incident and scattered compressional and shear wavefields according to a model of the compressional and shear wave velocities of propagation in the object's substructure.
